(19) United States
(12) Patent Application Publication
(10) Pub. No.: US 2026/0083457 A1
(43) Pub. Date: Mar. 26, 2026

(54) EMBOLIC COMPOSITIONS CONTAINING DISSIPATABLE ADDITIVE

(71) Applicant: Bard Peripheral Vascular, Inc., Franklin Lakes, NJ (US)

(72) Inventors: Jordan Addison, Gilbert, AZ (US); Hiep Quang Do, Chandler, AZ (US); Emily A. Goel, Phoenix, AZ (US); Michael A. Tycon, Scottsdale, AZ (US); Jeffrey Wang, Phoenix, AZ (US)

(73) Assignee: Bard Peripheral Vascular, Inc., Franklin Lakes, NJ (US)

(21) Appl. No.: 19/111,075

(22) PCT Filed: Sep. 14, 2022
(86) PCT No.: PCT/US22/76425

§ 371 Date: Mar. 12, 2025

(51) Int. Cl. A61B 17/12; A61B 17/00; A61L 24/00

(52) U.S. Cl. CPC A61B 17/12186; A61L 24/0015; A61L 24/0036; A61B 2017/00893; A61B 2017/1205

(57) ABSTRACT

Embolic compositions include a carrier liquid that solidifies to form a solid occlusion matrix at a target site in a blood vessel, and a dissipatable solid additive contained within the carrier liquid. The dissipatable solid additive includes microparticles that are substantially insoluble in the carrier liquid, that are dispersed in the matrix upon solidification of the matrix, and that are dissipated out of the matrix by action of blood flow in the blood vessel against the matrix. Thereby, the matrix develops a porous structure and the blood vessel at the target site is less than totally occluded after dissipation of the additive. Methods of occluding a blood vessel include injecting the embolic composition to a target site in a blood vessel, allowing the embolic composition to solidify, and allowing the dissipatable solid additive to be dissipated from the solid occlusion matrix by blood flow in the blood vessel.

### TECHNICAL FIELD

The present disclosure relates to liquid embolic compositions and, more particularly, to liquid embolic compositions containing a dissipatable additive that creates a porous embolic lattice.

### BACKGROUND

Embolization therapy is a minimally invasive surgery performed by interventional radiologists. Typical treatments may include entering the vasculature via a minor incision, such as in the arm or leg, and then extending devices such as guidewires and catheters through the vasculature to gain access to a treatment site, optionally aided by imaging techniques such as fluoroscopy. The embolic agent at the treatment site embolizes the vessel, blocking off the flow of blood downstream from the treatment site. When the embolization therapy is applied for tumor treatment, the blocking of blood flow downstream from the treatment site results in necrosis and/or shrinkage of the tumors.

The embolic agent of choice for embolization therapy depends on the desired clinical outcome, as well as the inherent properties of the embolic agent. Embolic agents commonly utilized in modern clinical settings generally belong to one of three classes: (1) mechanical structures such as coils or plugs that are lodged into portions of a vessel and occlude the vessel; (2) liquid embolics that are introduced at the desired occlusion site as a liquid or gel that solidifies in the vessel, thereby “casting” the structure; and (3) microparticles such as beads or irregular fragments that essentially agglomerate, group, or pack at an occlusion site to partially or totally block blood flow past the occlusion site.

Mechanical devices and liquid embolics are designed to create a complete occlusion, known as “total stasis.” These devices are typically promoted based on the speed of effecting total stasis, and with the goal of total stasis in view, any residual blood flow past the embolization point is considered a technical failure. Microparticles, on the other hand, enable control of the extent of stasis by altering particle size, physical compressibility or the particles, or total volume of the particles being injected to moderate the packing density within the vasculature. Owing to the level of control, microparticles have become the embolic agent of choice for procedures requiring controllable extent of stasis, distal embolization location, and treatment of small vessels.

Achieving the desired extent of blood flow occlusion is of high importance to an embolization procedure and varies based on the nature of the embolization treatment Procedures targeting end-organs, such as in benign prostatic hyperplasia treatment, frequently require total stasis. Other treatments involving inducing ischemia at structures within an organ, such as in uterine fibroid treatments, commonly target partial occlusion to prevent infarction of the remainder of the organ. Liver tumor embolization represents a complex scenario, in which the desired extent of stasis typically depends on the overall liver function, tumor manifestation, and preference of the skilled practitioner, with the end-point spanning total to partial stasis. In each of these scenarios, microparticles have been determined to be suitable.

Even so, control over the static endpoint depends on the physical characteristics of the embolic agent, combined with the practitioner's skill and experience in working with the embolic agent. Unlike procedures involving mechanical devices or liquid embolics, microparticle-based embolization procedures typically are characterized by slow particle injection to avoid over-embolization or particle reflux and by periodic waiting times to allow for particle packing and subsequent assessment of residual blood flow. Therefore, ongoing needs remain for embolic agents that can provide controllable levels of stasis without involving complex injection and waiting protocols.

### SUMMARY

Example embodiments disclosed herein are directed to embolic compositions. The embolic compositions include a carrier liquid that solidifies to form a solid occlusion matrix at a target site in a blood vessel; and dissipatable solid additive contained within the carrier liquid. The dissipatable solid additive includes microparticles that are substantially insoluble in the carrier liquid, that are dispersed in the solid occlusion matrix upon solidification of the solid occlusion matrix, and that are dissipated out of the solid occlusion matrix by action of blood flow in the blood vessel against the solid occlusion matrix. Thereby, the solid occlusion matrix develops a porous structure and the blood vessel at the target site is less than totally occluded after dissipation of the dissipatable solid additive.

Further example embodiments disclosed herein are directed to methods of occluding a blood vessel. The methods include injecting an embolic composition according to embodiments herein to a target site in a blood vessel; allowing the embolic composition to solidify to form a solid occlusion matrix; and allowing the dissipatable solid additive to be dissipated from the solid occlusion matrix by blood flow in the blood vessel and thereby develop a porous structure in the solid occlusion matrix. The methods may further include loading the embolic composition into a delivery device before injecting the embolic composition. The methods may further include preparing the embolic composition before loading the embolic composition, wherein preparing the embolic composition comprises mixing the carrier liquid and the dissipatable solid additive. The methods may further include determining a weight amount and a particle size of dissipatable solid additive to include in the embolic composition to achieve a desired extent of stasis in the blood vessel after dissipation of the dissipatable solid additive. Still further embodiments are directed to embolic compositions as described herein for use in a method of treatment by surgery. The method of treatment may include a step of occluding a blood vessel, preferably by any of the methods described herein.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a schematic of an embolic composition at time of injection at an occlusion site within a blood vessel.

FIG. 2 is a schematic of an occlusion site upon curing of the embolic composition.

FIG. 3 is a schematic of an occlusion site after a period of time has passed to enable blood to begin dissolving the dissipatable microparticles within the embolic lattice.

FIG. 4 is a schematic of an occlusion site after an additional period of time has passed, after which blood has dissolved a substantial portion of the dissipatable microparticles within the embolic lattice.

FIG. 5 is a schematic of an occlusion site after the dissipatable microparticles have been completely dissolved to leave behind an embolic lattice with a porous structure.

FIG. 6A is a schematic of an occlusion site with porosity created with a particular weight ratio of microparticles to liquid medium in the embolic composition.

FIG. 6B is a schematic of an occlusion site with porosity created with a weight ratio of microparticles to liquid medium in the embolic composition greater than the ratio of the embolic composition of FIG. 6A.

FIG. 7A is a schematic of an occlusion site in which the embolic composition includes microparticles that are dissipatable by melting at body temperature or through application of an external energy source.

FIG. 7B is a schematic of an occlusion site of FIG. 7A as the microparticles begin to melt.

FIG. 7C is a schematic of an occlusion site of FIG. 7A after the dissipatable microparticles have completely melted and removed from the embolic lattice, to leave behind an embolic lattice with a porous structure.

### DETAILED DESCRIPTION

The term “embolizing” as used in conjunction with “embolic compositions” and “embolic agents” refers to a process in which a material is injected into a blood vessel and the material thereafter fills or plugs the blood vessel and/or encourages clot formation so that blood flow through the vessel ceases.

The term “liquid embolic composition” refers to a fluid composition that is injected at a target site in a blood vessel where embolization is desired, then solidifies to fully or partially occlude the embolization site. Liquid embolics flow through the vasculature as a fluid, which allows for deep penetration of small diameter target vessels and the ability to conform to the lumen of any vasculature before transitioning into a solid material. Liquid embolics can be used to occlude vasculature of a variety of diameters.

The term “dissipatable” as used herein means a material capable of being dissipated or dispersed by action of a fluid flow against the material. Dissipation in this sense may involve an integral or unitary body of the material at a single location that is acted upon by the fluid flow, whereby portions of the unitary body or the entirety of the unitary body are dissipated or broken up, then carried away by the fluid flow. Specific examples of dissipation pertaining to embolic compositions herein are described subsequently in detail.

The terms “physiologically acceptable” and “pharmaceutically acceptable” refer to materials or compositions that are recognized as acceptable for introduction into vasculature of the human body in the amounts intended for introduction during an embolization procedure involving the embolic compositions of this disclosure.

Embolic compositions are described herein that contain a dissipatable additive, typically in the form of microparticles. The embolic compositions include an embolic carrier that solidifies at a target site in a blood vessel and initially totally occludes the vessel as a solid occlusion matrix. By action of blood flow against the solidified composition, the microparticles are dissipated from the solid occlusion matrix to leave voids in the solid occlusion matrix and result in a porous occlusion matrix. Upon formation of the porous occlusion matrix, the occlusion of the blood vessel is less than total. The amount of occlusion or stasis is controllable by tailoring the shapes and sizes of the microparticles and, thereby the shapes and sizes of the voids and pores left behind by the dissipation of the microparticles. The amount of occlusion or stasis is further controllable by choosing the weight fraction of the microparticles in the embolic composition prior to injection thereof. The ratio of carrier to microparticles may be chosen in view of the vessel size and the extent of stasis desired. For example, incorporation of a greater amount of microparticles, as a weight or volume percent of the embolic composition, will result in greater porosity and reduced stasis.

The combination of an embolic carrier, capable of complete distal stasis, with dissipatable additive microspheres to create a porous internal lattice structure, provides a single product that can span the technical and clinical requirements of a wide variety of embolization treatments. Employing a carrier embolic that casts the vasculature permits usage in small vasculature structures, eliminates migrations or off-target effects, and in some instances can be used to cover both the arterial and venous sides of a tumor in a manner not currently feasible with microparticles alone.

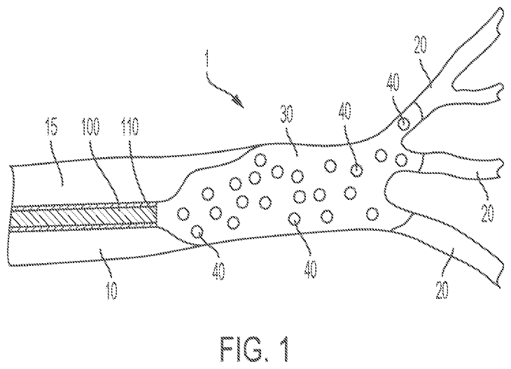

Concepts of the embolic compositions herein will now be described with reference to the drawings. Referring to FIG. 1, an embolic composition 30, 40 is delivered through a lumen 15 of a blood vessel 10 to a target site 1 of the blood vessel 10 by a delivery device 100 through a tip 110 of the delivery device 100. The embolic composition 30, 40 includes the carrier liquid 30 and microparticles 40 of the dissipatable solid additive. In the example of FIG. 1, the embolic composition 30, 40 flows even into capillary branches 20 of the blood vessel 10. The delivery device 100 then is withdrawn, and the embolic composition 30, 40 is allowed to solidify. Thereupon, a solid occlusion matrix is formed that is a combination of solidified carrier liquid and the dissipatable solid additive dispersed in the solidified carrier liquid.

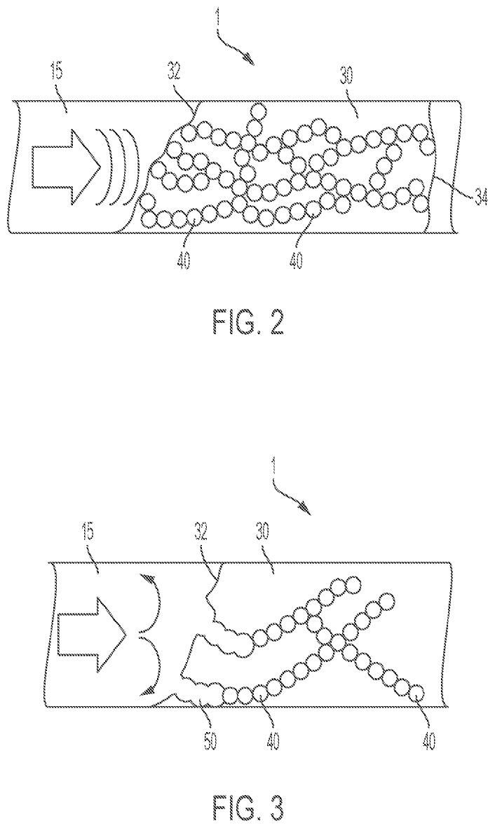

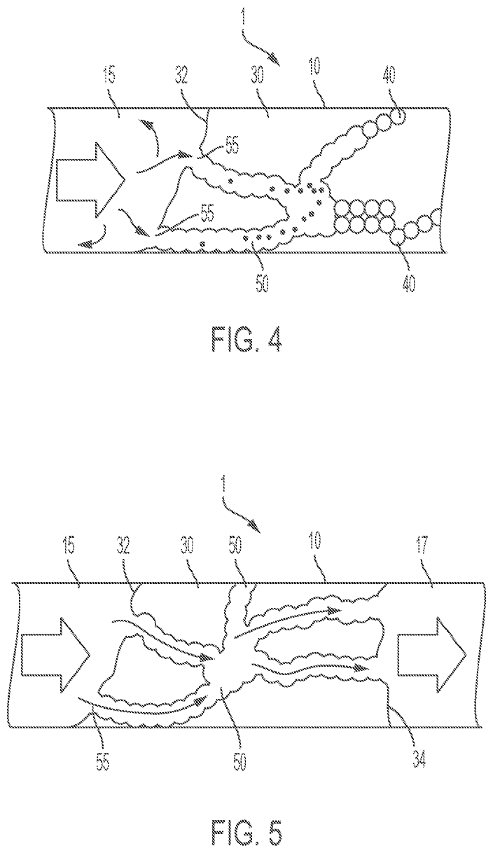

Referring to FIG. 2, after withdrawal of the delivery device, blood flowing through the lumen 15 (from left to right, as indicated in FIG. 2) encounters a proximal side 32 of the solid occlusion matrix 30, 40. Blood is initially unable to flow through the solid occlusion matrix 30, 40 to reach the distal side 34 thereof. Referring to FIG. 3, after a period of time, blood flow through the lumen 15 begins to dissipate microparticles 40 adjacent to the proximal side 32 to form a void 50 within the solid occlusion matrix. Referring to FIG. 4, after an additional period of time, the void 50 propagates in a distal direction by the action of blood flow against the proximal side 32 of the solid occlusion matrix. Though the void 50 has propagated, blood cannot yet pass entirely through the solid occlusion matrix, and a portion of the microparticles 40 of the dissipatable solid additive remain intact. Referring to FIG. 5, upon complete dissipation of the microparticles, the solid occlusion matrix has a defined pore structure made up of the voids 50 from where the microparticles 40 previously had been. Thereby, blood can flow through pore entrances 55, through the solid occlusion matrix from the proximal side 32 thereof to the distal side 34 thereof and into a distal portion 17 of the blood vessel.

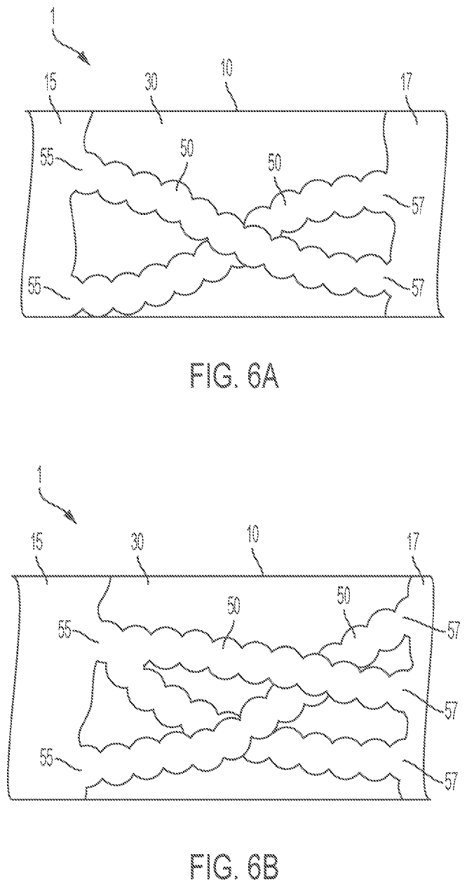

FIGS. 6A and 6B are comparable schematic diagrams of embolization sites 1, in which the solid occlusion matrix 30 after dissipation of microparticles has various amounts of pores 50, based on the amount of dissipatable solid additive present in the embolic composition prior to the solidification of the embolic composition. Particularly, the embolization site 1 of FIG. 6A has a solid occlusion matrix 30 with fewer pores 50, than the embolization site 1 of FIG. 6B, as a result of including a lesser amount by weight of dissipatable solid additive in the embolic composition injected at the embolization site 1 of FIG. 6A than in the embolic composition injected at the embolization site 1 of FIG. 6B. In both FIGS. 6A and 6B, blood in the lumen 15 of the blood vessel 10 flows into pore entrances 55 through the pores 50 to exit the solid occlusion matrix 30 via the pore exits 57 into the distal portion 17 of the blood vessel 10.

Reference will now be made in detail to various embodiments of embolic compositions. Methods of occluding blood vessels by use of the embolic compositions will be described subsequently.

The embolic compositions include a carrier liquid and a dissipatable solid additive contained within the carrier liquid. The carrier liquid is in a liquid state, and the dissipatable solid additive is in a solid state. The dissipatable solid in its solid state may be hard or soft, provided the dissipatable solid is present as a separate and discernable phase in the carrier liquid. The dissipatable solid additive may be suspended or dispersed in the carrier liquid. Though the embolic composition may include any physical mixture of the carrier liquid and the dissipatable solid additive, a substantially uniform suspension or dispersion of the dissipatable solid additive may be facilitated by mixing or agitation of the embolic composition, if required.

The carrier liquid of the embolic composition is a composition formulated to solidify and form a solid occlusion matrix at a target site in a blood vessel. The carrier liquid may be any known or to be developed liquid embolic formulation and is limited only with respect to the functional aspect of beginning in a substantially flowing or fluidic state prior to injection to the target site, then solidifying at the target site by any mechanism to form a solid occlusion matrix having the dissipatable additive present therein. The carrier liquid prior to injection may be a homogeneous liquid, a gel, a hydrogel, or a flowing suspension of a polymer in a physiologically acceptable solvent, for example. Homogeneous liquids, for example, may include monomer solutions that solidify at the target site by polymerizing.

Specific non-limiting examples of suitable carrier liquids and formulations will now be described.

One example of a suitable carrier liquid for the embolic compositions herein includes n-butyl cyanoacrylate (NBCA) of the Trufill® system, marketed by Cordis Neurovascular, Inc. In its monomer form, NBCA is a clear liquid that polymerizes to form a rigid matrix upon activation by contact with any ionic substance, such as blood, saline, ionic contrast media, or vascular endothelium. In commercial forms, the Trufill® system provides physicians with a glue kit consisting of NBCA, ethiodized oil, and tantalum powder, which are combined on site just prior to use. The polymerization of NBCA releases formaldehyde, leading to inflammation of the vessel wall and surrounding tissue, eventually causing a chronic granulomatous inflammation. The adhesive nature of NBCA allows it to mechanically occupy the intravascular lumen and stop blood flow regardless of blood coagulability. An example embolic composition of the present disclosure therefore may include a mixture of N-butyl cyanoacrylate and a dissipatable solid additive as described herein.

Another example of a suitable carrier liquid for the embolic compositions herein includes the Onyx® system of Medtronic. The Onyx® system is composed of ethylene vinyl alcohol (EVOH) copolymer dissolved in dimethyl sulfoxide (DMSO), optionally with a tanalum-based additive for visualization by radiography. Onyx® functions by forming a solid EVOH precipitate as the DMSO carrier solvent dissipates as it comes into contact with the ionic contents of the bloodstream, causing the polymer to progressively solidify by precipitation. Unique among liquid embolics, Onyx® solidifies in an “outside-in” fashion. This results in the almost instantaneous formation of a solid cast around the exterior of the flow, while the interior remains fluid and continues to flow deeper into the lesion. Generally, regardless of the formulation, Onyx® completely solidifies within five minutes of injection. An example embolic composition of the present disclosure therefore may include a mixture of EVOH and DMSO, in combination with a dissipatable solid additive as described herein.

Another example of a suitable carrier liquid for the embolic compositions herein includes the non-adhesive liquid embolic agent PHIL (Precipitating Hydrophobic Injectable Liquid), manufactured by Microvention. Like the Onyx® system, PHIL precipitates and solidifies as it comes into contact with the ionic content of the blood stream and DMSO progressively dissipates. PHIL™ is a non-adhesive embolic suspended in DMSO, including poly(lactide-co-glycolide) copolymer and poly(hydroxyl ethyl methacrylate), with triiodophenol incorporated into a portion of the monomers via a covalent linkage, providing radiopacity for visualization. PHIL™ remains suspended in DMSO until it comes into contact with blood or water, at which time the polymer precipitates into a solid. Solidification is very rapid, on the order of a few minutes. An example embolic composition of the present disclosure therefore may include a mixture of poly(lactide-co-glycolide) copolymer and poly(hydroxyl ethyl methacrylate) in DMSO, in combination with a dissipatable solid additive as described herein.

Another example of a suitable carrier liquid for the embolic compositions herein includes Histoacryl (B. Braun, Germany) and Glubran (GEM, Italy), which solidify by polymerization. Glubran is a synthetic surgical glue, CE certificated, for internal and external use, with haemostatic, adhesive, sealer and bacteriostatic properties. When used in a moist environment, it quickly polymerizes into a thin elastic film that has high tensile strength and firmly adheres to the anatomy of the tissue on which it is applied. Once polymerized, Glubran acts as a bioinert material that is used in open and laparoscopic surgery, as well as in endovascular surgery as an embolic agent. Histoacryl consists of monomeric n-butyl-2-cyanoacrylate, which polymerizes quickly in contact with tissue fluid. Glubran is a combination of n-butyl-2-cyanoacrylate and methacryloisosulfolane. An example embolic composition of the present disclosure therefore may include Histoacryl or Glubran in combination with a dissipatable solid additive as described herein. An example embolic composition of the present disclosure therefore may include n-butyl-2-cyanoacrylate, methacryloisosulfolane, and a dissipatable solid additive as described herein.

Another example of a suitable carrier liquid for the embolic compositions herein includes the GPX Embolic Device by Fluidx. This system incorporates a proprietary system based on biomaterials from sandcastle worms that includes an aqueous-based, low-viscosity polymer solution that precipitates by an ionic exchange in solution to form a durable gel-like solid, without DMSO or polymerization. The compositions include an in situ solidifying complex coacervate composed of a polycation, a polyanion, and a salt that produces ions in water. Example polycations include polyguanidinyl copolymers such as polyguanidinyl acrylates, polyguanidinyl methacrylates, polyguanidinyl acrylamides, or polyguanidinyl methacrylamides, salmine, or clupein. Example polyanions include polyphosphates such as polyphosphoserine. Example salts include sodium chloride, sodium acetate, or sodium carbonate. An example embolic composition of the present disclosure may include a composition of the GPX Embolic Device containing the polycation, polyanion, and salt in combination with a dissipatable solid additive as described herein.

Another example of a suitable carrier liquid for the embolic compositions herein includes Embrace from Instylla. The Embrace system consists of two low-viscosity liquid precursors that polymerize intravascularly when simultaneously injected into blood vessels to form a soft, water-based polyethylene glycol (PEG) hydrogel that liquefies by hydrolysis over approximately six months. For the Embrace system, a first liquid comprising an initiator is delivered through a first catheter lumen to a target lumen, then a second liquid is delivered that comprises a co-initiator through a second catheter lumen to the target lumen. At least one of the first liquid and the second liquid includes a water soluble polymer that comprises a plurality of functional groups. The initiator and the co-initiator react with each other to form a radical initiator that initiates a free radical polymerization of the water soluble polymer functional groups to crosslink the water soluble polymer to form an embolization material in the target lumen when the first liquid and the second liquid mix with each other. Example water soluble polymers include polysaccharides, hyaluronic acids, proteins, peptides, poly(ethylene glycol) s, or polyvinyl alcohols. Example initiators include peroxides. An example embolic composition of the present disclosure therefore may include the water soluble polymers and the initiator components of the Embrace system, in which at least one of the two components contains a dissipatable solid additive as described herein.

Another example of a suitable carrier liquid for the embolic compositions herein includes the liquid embolic from Theratarget including silk elastin protein polymers (SELP). The SELP material is a liquid at room temperature but at the human body temperature of 37° C., it transitions into a solid hydrogel capable of vessel occlusion. An example embolic composition of the present disclosure therefore may include silk elastin protein polymers in combination with a dissipatable solid additive as described herein. A further example embolic composition of the present disclosure may include the SELP liquid composition, in combination with a dissipatable solid additive as described herein.

Another example of a suitable carrier liquid for the embolic compositions herein includes shear-thinning compositions that thin when a force is applied to the carrier liquid during injection but that, when the force is removed, becomes a soft solid. Examples of such shear-thinning compositions are disclosed in international application publication WO 2022/036322 A1. Each and every composition described in WO 2022/036322 A1 can be used in combination with a dissipatable solid additive as described herein for the present invention. An example embolic composition of the present disclosure may include gelatin, a nanosilicate, and a dissipatable solid additive as described herein. A further example embolic composition of the present disclosure may include gelatin, a nanosilicate, a radiopaque contrast agent, and a dissipatable solid additive as described herein. Examples of nanosilicates include, without limitation, lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets).

The dissipatable solid additive of the embolic compositions herein are microparticles. The microparticles are substantially insoluble in the carrier liquid and, thus, are introduced into the body as solid components of the embolic composition in the carrier liquid. As used herein, “substantially insoluble in the carrier liquid” means that the dissipatable solid additive is either completely insoluble in the carrier liquid or has a solubility in the carrier liquid that is significantly less than its solubility in an aqueous medium such as blood, whereby during a treatment procedure the dissipatable solid additive remains sufficiently intact while the solid occlusion matrix is forming to enable formation of voids in the solid occlusion matrix after the carrier liquid solidifies.

When the carrier liquid solidifies to form a solid occlusion matrix, the microparticles are dispersed in the solid occlusion matrix. Over a period of time at the target site, the microparticles are dissipated out of the solid occlusion matrix by action of blood flow in the blood vessel against the solid occlusion matrix. As the dissipatable solid additive is dissipated out of the solid occlusion matrix, the solid occlusion matrix develops a porous structure. Thereby, the blood vessel at the target site is less than totally occluded after dissipation of the dissipatable solid additive, because blood can flow through the porous structure of the solid occlusion matrix, though to a lesser extent than the blood would be able to flow in the absence of the solid occlusion matrix. The porous structure may have the form of interconnected voids within the solid occlusion matrix. The porous structure is significantly voluminous to enable blood to pass through the solid occlusion matrix from a proximal side thereof to a distal side thereof but is not so extensive as to compromise the mechanical integrity of the solid occlusion matrix and cause the solid occlusion matrix to disintegrate at the target site. The extent of partial occlusion of the blood vessel at the target site is tunable based on the selection of the dissipatable solid additive, particularly with regard to the material of the dissipatable solid additive; the size, size distribution, and shape of the microparticles of the dissipatable solid additive; and the melting characteristics of the dissipatable solid additive.

The microparticles of the dissipatable solid additive generally may have any shape or size chosen to result in a porous structure in the solid occlusion matrix that has a desired extent of stasis. For example, the microparticles may be spherical, cylindrical, cubic, elliptical, irregularly shaped, or oblong. The microparticles may have particle sizes, as determined by laser diffraction, from 10 μm to 1200 μm, or from 10 μm to 1000 μm, or from 10 μm to 500 μm, or from 50 μm to 500 μm, or from 100 μm to 500 μm, or from 200 μm to 400 μm, or from 250 μm to 350 μm, or from 10 μm to 400 μm, or from 10 μm to 300 μm, or from 10 μm to 200 μm, or from 10 μm to 100 μm, for example. The particle sizes of spherical microparticles are defined by their diameter. The particle sizes of other shapes of microparticles are defined by an average of the particle's greatest length, greatest height, and greatest depth. Different vessel diameters coupled with the desired extent of occlusion may benefit from non-spherical geometries to ensure adequate connectivity of the lattice voids while maintaining sufficient integrity of the overall embolization occlusion.

In some examples, the dissipatable solid additive includes or consists of microparticles of a compound soluble in blood. Non-limiting examples of suitable compounds soluble in blood include any physiologically acceptable salt, sugars, starches, sugar alcohols (such as sorbitol, xylitol, mannitol, etc.) and water soluble polymers, such as casein sodium, polyvinyl alcohol, poly ethylene glycol, polyvinyl pyrrolidone, polyacrylic acid, N-(2-hydroxypropyl) methacrylamide, sodium carboxymethylcellulose, polyglycolide (PGA), poly(D,L-lactide-co-glycolide) (PLGA), polycaprolactone (PCL), and polylactic acid (PLA, PDLA, PLLA). A specific non-limiting example of physiologically acceptable salts includes sodium chloride.

The dissipation of the dissipatable solid additive that is soluble or degradable in blood occurs by the dissolving or degrading of the dissipatable solid additive in the blood or a component of the blood and, thereafter, being washed away from the solid occlusion matrix by the flowing of the blood against the solid occlusion matrix or any porous structure that has begun to develop from dissipation of an initial portion of the dissipatable solid additive.

In further examples, the dissipatable solid additive includes or consists of microparticles of a physiologically acceptable material that is solid at clinical temperatures prior to injection (such as at room temperature of 25° C.±3° C.) and that softens or melts at a body temperature in the blood vessel (such as at the normal human body temperature of 37° C., for example) or upon application of an external energy source. An example external energy source includes ultrasound, whereby a practitioner may administer ultrasound radiation from outside the body to cause the dissipatable solid additive, which is already at the target site inside the body, to soften or melt.

Non-limiting examples of suitable physiologically acceptable materials that soften or melt at a body temperature in the blood vessel or upon application of an external energy source include fatty acids, fatty acid esters, fatty alcohols, hydrocarbons, solid lipids, and various hydrocarbons. Examples of suitable fatty acids include caprylic acid, capric acid, lauric acid, stearic acid, and combinations thereof. Examples of suitable fatty acid esters include hydrogenated palm kernel oil, lanolin, triglyceride esters, hydrogenated vegetable oils, hydrogenated soybean oil, hydrogenated coconut oil, hydrogenated cotton oil, methylstearate, and combinations thereof. Examples of suitable fatty alcohols include 1-tridecanol, 1-dodecanol, cetyl alcohol, and combinations thereof. Examples of suitable solid lipids include cacao butter. Examples of suitable hydrocarbons include paraffin and petroleum jelly.

The dissipation of the dissipatable solid additive that is solid at clinical temperatures prior to injection and that softens or melts at a body temperature in the blood vessel occurs as a result of the dissipatable solid additive mechanically detaching from the solid occlusion matrix upon softening or melting. The mechanical detachment, in turn, enables the strength of blood flow to wash away the softened or melted additive from the solid occlusion matrix by the flowing of the blood against the solid occlusion matrix or any porous structure that has begun to develop from dissipation of an initial portion of the softened or melted additive.

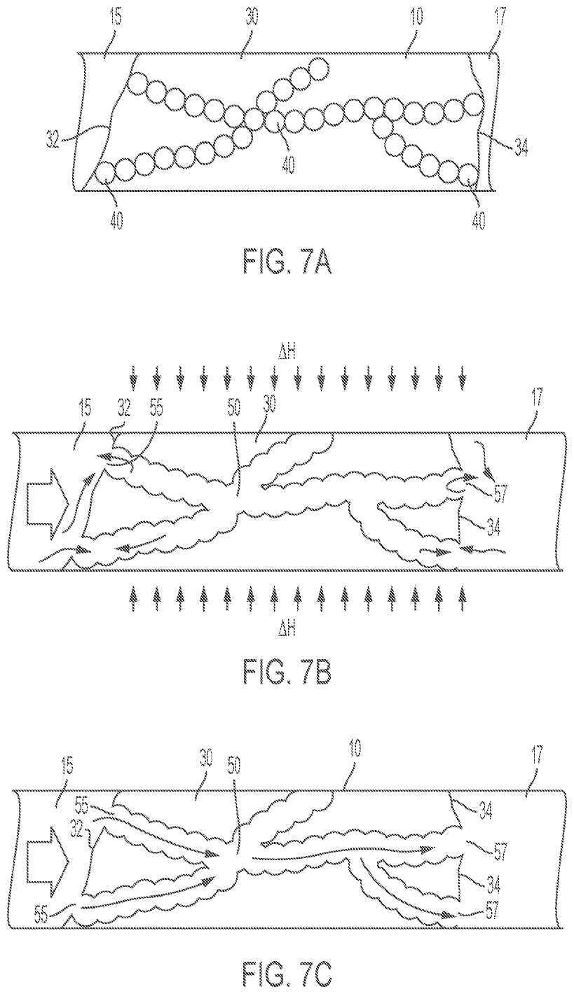

Referring to FIGS. 7A-7C, the solid occlusion matrix 30 includes microparticles 40 of a material that softens or melts at a body temperature in the blood vessel 10 or upon application of an external energy source. FIG. 7A depicts the initial solid occlusion matrix 30 immediately after injection and solidification of the embolic composition. The solid occlusion matrix 30 has a proximal side 32 first encountered by blood flowing in the lumen 15 of the blood vessel, microparticles 40 dispersed within the solid occlusion matrix 30, and a distal side 34 adjacent to a distal portion 17 of the blood vessel 10. FIG. 7B depicts heat flow (AH) into the blood vessel 10, either from the body itself or applied from an external source. The heat flow causes the microparticles 40 to soften or melt and, thereby, to mechanically detach from the solid occlusion matrix 30. Blood flows to the proximal side 32 of the solid occlusion matrix and begins to dissipate, remove, or wash out the softened material to form pores 50 that propagate from pore entrances 55 to pore exits 57 at the distal side 57 of the solid occlusion matrix 30. FIG. 7C depicts the solid occlusion matrix 30 after the complete removal of the dissipatable solid additive. In this final state, blood flow in the blood vessel 10 from the lumen 15 to the distal side 17 of the lumen is partially occluded in that blood traverses through the solid occlusion matrix 30 through the pores 50 from pore entrances 55 at the proximal side 32 of the solid occlusion matrix 30 to pore exits 57 at the distal side 34 of the solid occlusion matrix 30.

In further examples, the dissipatable solid additive includes or consists of microparticles that swell in the carrier liquid prior to placement at the target site and shrink upon contact with blood after placement at the target site. Examples of such materials include starches, gelatins, gluten, chitin, or combinations thereof.

The embolic compositions herein include an amount by weight dissipatable solid additive sufficient to provide enough volume of dissipatable solid additive within the solid occlusion matrix, such that when the dissipatable solid additive dissipates, a pore structure develops that enables some amount of blood flow through the solid occlusion matrix. Moreover, the amount of dissipatable solid additive should not be so great that the mechanical stability of the solid occlusion matrix is severely compromised when the solid additive dissipates. In some examples, the embolic compositions may include from 5% to 95% by weight dissipatable solid additive. In other examples, the embolic compositions herein may include from 20% to 80% by weight dissipatable solid additive, or from 30% to 80% by weight dissipatable solid additive, or from 40% to 80% by weight dissipatable solid additive, or from 40% to 70% by weight dissipatable solid additive, or from 40% to 60% by weight dissipatable solid additive, based on the total weight of the embolic composition. It should be understood that the embolic compositions may include less than 5% by weight or greater than 95% by weight dissipatable solid additive, as long as a pore structure may be formed without collapsing the solid occlusion matrix at the target site.

The embolic compositions herein may further include, as a component of the carrier liquid or the dissipatable solid additive, a contrast agent, a therapeutic agent, or combination thereof. The contrast agent may be any contrast agent commonly used for embolization procedures to enable a practitioner to view the position and extent of embolization by fluoroscopy, radioscopy or other technique. The therapeutic agent may be a drug or radioactive element or compound suitable for use in embolization therapy for the benefit of an affected organ.

In some example embolic compositions, the carrier liquid includes a physiologically acceptable solvent and a polymer dissolved in the physiologically acceptable solvent. In such compositions, removal of the physiologically acceptable solvent by blood flow causes the polymer to precipitate to form the solid occlusion matrix. In non-limiting examples, the physiologically acceptable solvent is chosen from dimethylsulfoxide or physiologically acceptable alcohols. In non-limiting examples, the polymer is chosen from poly(lactide-co-glycolide), poly(hydroxymethylmethacrylate), poly(vinyl alcohol), ethylene vinyl alcohol, combinations thereof, or copolymers thereof.

A porous occlusion matrix thus may be formed from the embolic compositions described herein. The porous occlusion matrix includes a polymer having a pore structure defined therein that provides fluidic communication from a proximal side of the porous occlusion matrix to a distal side of the porous occlusion matrix.

The embolic compositions herein may be used to occlude blood vessels in a clinical embolization treatment. Accordingly, methods of occluding a blood vessel may include injecting an embolic composition as described herein to a target site in a blood vessel. Then, the embolic composition is allowed to solidify to form a solid occlusion matrix. Then, the dissipatable solid additive is allowed to be dissipated from the solid occlusion matrix by blood flow in the blood vessel and thereby develop a porous structure in the solid occlusion matrix.

The methods of occluding a blood vessel may further include loading the embolic composition into a delivery device before injecting the embolic composition. Example delivery devices include embolization catheters. The methods of occluding a blood vessel may further include preparing the embolic composition before loading the embolic composition, wherein preparing the embolic composition comprises mixing the carrier liquid and the dissipatable solid additive.

The methods of occluding a blood vessel may further include determining a weight amount and a particle size of dissipatable solid additive to include in the embolic composition to achieve a desired extent of stasis in the blood vessel after dissipation of the dissipatable solid additive, then mixing the carrier liquid and the dissipatable solid additive according to the determined amounts or proportions.

Determinations that may be applicable to the preparation of the embolic composition include particle size of the dissipatable solid additive, particle geometry of the dissipatable solid additive, the vessel diameter at the target site, the vessel blood-flow rate at the target site, and the desired level of occlusion. Regarding particle size, larger diameter vessels may require larger microparticles to achieve sufficient porosity for an interconnected lattice to form. Regarding particle geometry, nonspherical particles may be able to achieve greater interstitial fluidic porosity at a lower particle incorporation ratio. Regarding vessel diameter at the target site, large diameter vessels may require greater incorporation of the dissolving microparticles into the carrier embolic to achieve sufficient porosity for an interconnected lattice to form. Regarding vessel blood-flow rate, vessels with higher flow rates or increased blood pressure may require lower microparticle loading to achieve the desired stasis endpoint compared to vessels with slower blood flow or reduced blood pressure. Regarding desired level of occlusion, reduced microparticle loading would result in a reduced porosity, leading to a higher level of stasis. Lower levels of stasis would be accomplished by a greater incorporation of the microparticles, resulting in increased porosity.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. An embolic composition comprising:
a carrier liquid that solidifies to form a solid occlusion matrix at a target site in a blood vessel; and
a dissipatable solid additive contained within the carrier liquid,
wherein:
the dissipatable solid additive comprises microparticles that are substantially insoluble in the carrier liquid, that are dispersed in the solid occlusion matrix upon solidification of the solid occlusion matrix, and that are dissipated out of the solid occlusion matrix by action of blood flow in the blood vessel against the solid occlusion matrix, whereby the solid occlusion matrix develops a porous structure and the blood vessel at the target site is less than totally occluded after dissipation of the dissipatable solid additive.

2. The embolic composition of claim 1, wherein the dissipatable solid additive comprises microparticles of a compound soluble in blood.

3. The embolic composition of claim 2, wherein the compound soluble in blood is a physiologically acceptable salt, a sugar, a starch, a sugar alcohol, or a water-soluble polymer.

4. The embolic composition of claim 2, wherein the compound soluble in blood is chosen from sodium chloride, sorbitol, or polyvinyl pyrrolidone.

5. The embolic composition of claim 1, wherein the dissipatable solid additive comprises microparticles of a physiologically acceptable material that softens or melts at a body temperature in the blood vessel or by application of an external energy source.

6. The embolic composition of claim 5, wherein the physiologically acceptable material that softens or melts at a body temperature in the blood vessel is chosen from fatty acids, fatty acid esters, fatty alcohols, solid lipids, and hydrocarbons.

7. The embolic composition of claim 1, wherein the dissipatable solid additive comprises microparticles that swell in the carrier liquid prior to placement at the target site and shrink upon contact with blood after placement at the target site.

8. The embolic composition of claim 1, wherein the embolic composition comprises from 20% to 80% by weight dissipatable solid additive, based on the total weight of the embolic composition.

10. The embolic composition of claim 1, wherein the microparticles have particle sizes from 10 μm to 500 μm.

11. The embolic composition of claim 1, wherein the microparticles have particle sizes from 250 μm to 350 μm.

12. The embolic composition of claim 1, further comprising, as a component of the carrier liquid or the dissipatable solid additive, a contrast agent, a therapeutic agent, or combination thereof.

13. The embolic composition of claim 1, wherein:
the carrier liquid comprises a physiologically acceptable solvent and a polymer dissolved in the physiologically acceptable solvent; and
removal of the physiologically acceptable solvent by blood flow causes the polymer to precipitate to form the solid occlusion matrix.

14. The embolic composition of claim 13, wherein:
the physiologically acceptable solvent comprises dimethylsulfoxide; and
the polymer is chosen from poly(lactide-co-glycolide),
poly(hydroxymethylmethacrylate), poly(vinyl alcohol), ethylene vinyl alcohol, combinations thereof, or copolymers thereof.

15. A porous occlusion matrix formed from the embolic composition of claim 1, the porous occlusion matrix comprising a polymer having a pore structure defined therein that provides fluidic communication from a proximal side of the porous occlusion matrix to a distal side of the porous occlusion matrix.

16. A method of occluding a blood vessel, the method comprising:
injecting an embolic composition according to claim 1 to a target site in a blood vessel;
allowing the embolic composition to solidify to form a solid occlusion matrix; and
allowing the dissipatable solid additive to be dissipated from the solid occlusion matrix by blood flow in the blood vessel and thereby develop a porous structure in the solid occlusion matrix.

17. The method of claim 16, further comprising loading the embolic composition into a delivery device before injecting the embolic composition.

18. The method of claim 17, further comprising preparing the embolic composition before loading the embolic composition, wherein preparing the embolic composition comprises mixing the carrier liquid and the dissipatable solid additive.

19. The method of claim 18, further comprising determining a weight amount and a particle size of dissipatable solid additive to include in the embolic composition to achieve a desired extent of stasis in the blood vessel after dissipation of the dissipatable solid additive.

20. (canceled)
